(19) United States
(12) Patent Application Publication
(10) Pub. No.: US 2008/0189882 A1
(43) Pub. Date: Aug. 14, 2008

(54) Hot-Light Resistant Blue Dispersion Dyes

(75) Inventors: Hartwig Jordan, Bergisch-Gladbach (DE); Andreas Endres, Leverkusen (DE)

Correspondence Address: CONNOLLY BOVE LODGE & HUTZ, LLP, P O BOX 2207, WILMINGTON, DE, 19899, US

(73) Assignee: DyStar Textifarben GmbH & Co. Deutschland, Frankfurt am Main (DE)

(21) Appl. No.: 11/915,661

(22) PCT Filed: May 30, 2006
(86) PCT No.: PCT/EP2006/062721

§ 371 Date: Nov. 27, 2007

(30) Foreign Application Priority Data
Jun. 2, 2005 (DE) 10 2005 025 276.2

(51) Int. Cl. C09B 67/22; C07D 221/18; C09B 5/42

(52) U.S. Cl. 8/639; 546/59; 8/643

(57) ABSTRACT

The present invention relates to dyes of the general formula I

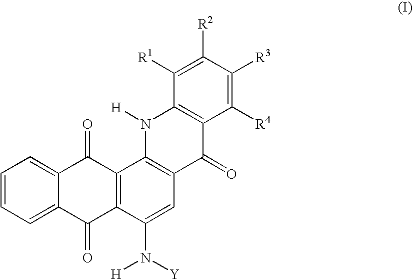

where R¹ to R⁴ and Y are each as defined in claim 1, dye mixtures comprising same, processes for their preparation, and also their use for dyeing and printing hydrophobic synthetic materials.

The present invention relates to the field of disperse dyes.

Polyester fibers for use in automotive fabrics are generally dyed blue using dyes of the class of the anthraquinones. However, the dyes of this type which are used in present-day commercial practice do not fully satisfy high requirements with regard to lightfastness, especially the colorfastness to light at high temperatures. This applies in particular to combinations (known as trichromats) comprising yellow and red disperse dyes colorfast to light at high temperatures, where it is important that the individual components of the trichromat fade at the same rate in order that there are no hue changes under the action of light.

Anthraquinoneacridones and their use as dyes are already known from the literature. For instance, DE239543, CH56472, CH144867, DE579326, DE665598, U.S. Pat. No. 2,185,140, DE652773 describe vat dyes of this type for dyeing cotton.

But anthraquinoneacridones have also already been described for dyeing polyester fibers. See DE 1 176 775 B, but in particular WO02/051942, WO02/051924, DE 1 171 101 B and DE 1 278 391 B. The latter describes a process for dyeing and printing fiber composed of high molecular weight polyesters with, for example, anthraquinonone-3,4-benzacridones bearing a butyrylamino or β-chloropropionylamino radical in position 1. The dyes do indeed provide strong dyeings having excellent fastness properties, but are deficient with regard to affinity for polyester.

It is an object of the present invention to provide blue-dyeing disperse dyes that are superior to existing dyes with regard to colorfastness to light at high temperatures, especially in admixtures with other dyes in a trichromat, and also with regard to their affinity.

We have found that this object is achieved, surprisingly, by specifically selected representatives from the series of the anthraquinoneacridones.

The present invention thus provides dyes of the general formula I

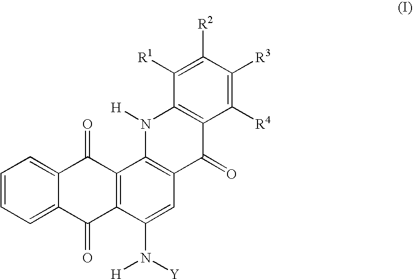

where
R¹ to R⁴ are independently hydrogen, (C₁-C₄)-alkyl, (C₁-C₄)-alkoxy, CF₃, NO₂, CN, halogen, COR⁵, COOR⁵, CONR⁶R⁷, SO₂R⁵ or SO₂NR⁶R⁷,
   where R⁵, R⁶ and R⁷ are each hydrogen or (C₁-C₄)-alkyl, but R⁶ and R⁷ cannot both be hydrogen; and
Y is —CO(CH₂)₃Cl or —SO₂R⁸,
   where R⁸ is (C₁-C₈-alkyl, (C₁-C₈)-alkyl, substituted by NO₂, CN, halogen or phenyl, phenyl, phenyl substituted by one or more substituents selected from (C₁-C₄)-alkyl, (C₁-C₄)-alkoxy, CF₃, NO₂, CN, halogen, COR⁵, COOR⁵, CONR⁶R⁷, SO₂R⁵ and SO₂NR⁶R⁷, or is naphthyl or naphthyl substituted by one or more substituents selected from
   (C₁-C₄)-alkyl, (C₁-C₄)-alkoxy, CF₃, NO₂, CN, halogen, COR⁵, COOR⁵, CONR⁶R⁷, SO₂R⁵ and SO₂NR⁶R⁷,
      although R⁸ cannot be 4-methylphenyl when R¹ to R⁴ are all hydrogen and cannot be phenyl or 4-methylphenyl when R¹ and R³ are both chlorine and R² and R⁴ are both hydrogen.

(C₁-C₄)-Alkyl R¹ to R⁷ may each be linear or branched and are for example methyl, ethyl, n-propyl, i-propyl, n-butyl, i-butyl, sec-butyl or tert-butyl. (C₁-C₈)-Alkyl R⁸ may additionally be selected from pentyl, hexyl, heptyl and octyl Methyl and ethyl are particularly preferred alkyl. The same holds mutatis mutandis for (C₁-C₄)-alkoxy groups, for which methoxy and ethoxy are accordingly particularly preferred.

Halogen is for example fluorine, chlorine or bromine, with chlorine and bromine being preferred.

R¹ to R⁴ are each preferably hydrogen.

Examples of R⁸ are particularly ethyl, n-propyl, i-propyl, n-butyl, 1-naphthyl, 2-naphthyl, phenyl, 4-methylphenyl, 4-chlorophenyl, 2-bromophenyl, 4-bromophenyl, 2-nitrophenyl, 3-nitrophenyl, 4-nitrophenyl, phenylmethyl, 4-chloro-3-nitrophenyl, 3-trifluoromethylphenyl, 3,4-dimethoxyphenyl and 4-methoxyphenyl.

The present invention's dyes of the general formula I may be utilized together with one or more dyes of the kind typically used for dyeing polyesters fibers or polyester textile materials for automotive fabrics.

The present invention accordingly also provides dye mixtures comprising at least one dye of the general formula I and at least one dye useful for dyeing polyester textile materials for automotive fabrics.

Dyes useful for dyeing polyester textile materials for automotive fabrics are in particular azo, disazo, anthraquinone, nitro and naphthalimide dyes, which will be well known to those skilled in the art.

Preferred yellow and orange dyes of this kind are for example the Colour Index listings C.I. Disperse Yellow 23, 42, 51, 59, 65, 71, 86, 108, 122, 163, 182 and 211, C.I. Solvent Yellow 163, C.I. Disperse Orange 29, 30, 32, 41, 44, 45, 61 and 73, C.I. Pigment Orange 70, C.I. Solvent Brown 53, and also dyes of the formulae II and III

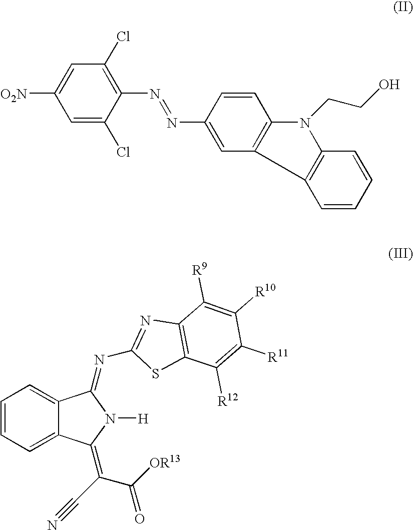

where
R⁹ to R¹² are independently hydrogen, chlorine, methyl, ethyl, isopropyl, tert-butyl, cyclohexyl, methoxy, ethoxy, n-propoxy, n-butoxy, methoxyethyl, ethoxyethyl, butoxyethyl or phenoxy, and
R¹³ is methyl, ethyl, propyl, isopropyl, allyl, n-butyl, isobutyl, n- and isopentyl, hexyl, octyl, 2-ethylhexyl, methoxyethyl, ethoxyethyl, butoxyethyl, butoxyethoxyethyl.

Preferred red dyes of this kind are for example the Colour Index listings C.I. Disperse Red 60, 82, 86, 91, 92, 127, 134, 138, 159, 167, 191, 202, 258, 279, 284, 302 and 323, C.I. Solvent Red 176, and also dyes of the formulae IV, V and VI

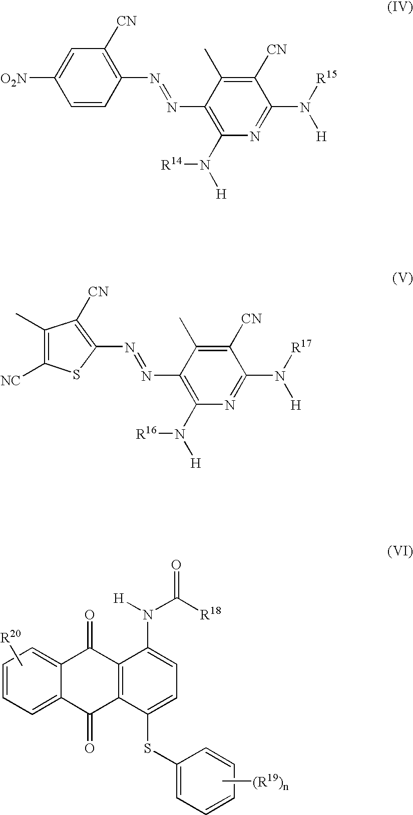

where
R¹⁴ and R¹⁵ are independently hydroxyethoxyethyl or phenyl,
R¹⁶ and R¹⁷ are independently hydrogen, hydroxyethoxyethyl, hydroxybutoxypropyl, acetoxyethoxyethyl or acetoxybutoxypropyl,
R¹⁸ is (C₁-C₈)-alkyl, phenyl or phenyl substituted by (C₁-C₄)-alkyl, hydroxyl or halogen, and
R¹⁹ and R²⁰ are independently hydrogen or halogen, and also
n is 0, 1 or 2.

Preferred blue and violet dyes of this kind are for example the Colour Index listings C.I. Blue 27, 54, 56, 60, 73, 77, 79, 79:1, 87, 266, 333 and 361, C.I. Disperse Violet 27, 28, 57 and 95 and also the dyes of the formula VII

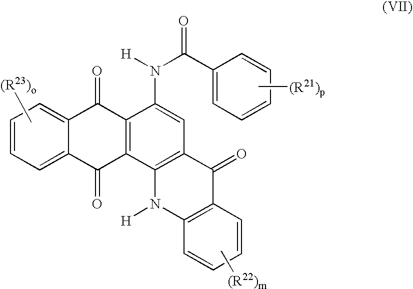

where
R²¹, R²² and R²³ are independently (C₁-C₈)-alkyl, halogen or hydroxyl, and
m, o and p are independently 0, 1 or 2.

In the dye mixtures of the present invention, the fractions of dye or dyes of the general formula I and of dye or dyes useful for dyeing polyester textile materials for automotive fabrics depend solely on the hue to be achieved, and thus may vary within wide limits. In general, the amounts of dye or dyes of the general formula I range from 1% to 99% by weight and the amounts of dye or dyes useful for dyeing polyester textile materials for automotive fabrics from 99% to 1% by weight.

The present invention's dyes of the general formula I are obtainable in a conventional manner.

For instance, they are obtainable by reacting a compound of the general formula VIII

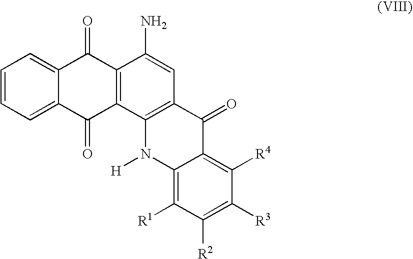

where R¹ to R⁴ are each as defined above, with a compound of the general formula IX

Hal-Y (IX)

where Hal is halogen, particularly chlorine, and Y is as defined above.

This reaction can be carried out with or without the assistance of acid-binding agents familiar to one skilled in the art.

The compounds of the general formula VII are obtainable for example by reacting bromamine acid of the formula X

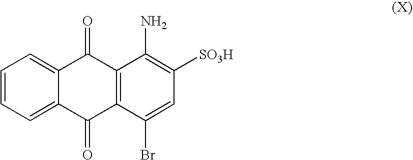

with a substituted anthranilic acid of the general formula XI

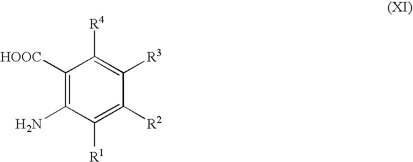

where R¹ to R⁴ are each as defined above, to form the compound of the general formula XII

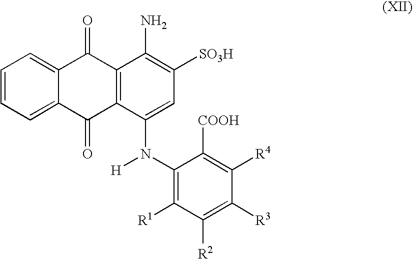

and cyclizing the latter by means of chlorosulfonic acid to form the compound of the general formula (XIII)

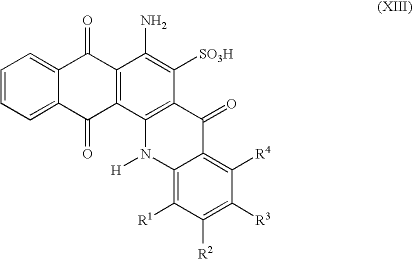

Finally, the sulfonic acid group is removed, for example with sodium dithionite.

The reaction of bromamine acid of the formula X with a substituted anthranilic acid of the general formula XI preferably takes place in the presence of copper powder and of a base under otherwise well-known reaction conditions. The other reaction steps mentioned are also carried out under well-known reaction conditions.

The present invention's dyes and dye mixtures are very useful for dyeing and printing hydrophobic synthetic materials, the dyeings and prints obtained having a remarkably high lightfastness and colorfastness to light at high temperatures, so that the textiles thus dyed can be used for automotive interiors.

The dyes of the present invention particularly exhibit a better build-up performance than the dyes of WO02/051942 and are also superior to those dyes in terms of colorfastness in pale shades when exposed to light at high temperatures.

The present invention thus also provides for the use of dyes of the general formula I

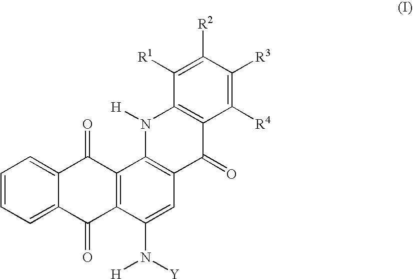

where
R¹ to R⁴ are independently hydrogen, (C₁-C₄)-alkyl, (C₁-C₄)-alkoxy, CF₃, NO₂, CN, halogen, COR⁵, COOR⁵, CONR⁶R⁷, SO₂R⁵ or SO₂NR⁶R⁷,
   where R⁵, R⁶ and R⁷ are each hydrogen or (C₁-C₄)-alkyl, but R⁶ and R⁷ cannot both be hydrogen; and
Y is —CO(CH₂)₃Cl or —SO₂R⁸,
   where R⁸ is (C₁-C₈)-alkyl, (C₁-C₈)-alkyl, substituted by NO₂, CN, halogen or phenyl, phenyl, phenyl substituted by one or more substituents selected from (C₁-C₄)-alkyl, (C₁-C₄)-alkoxy, CF₃, NO₂, CN, halogen, COR⁵, COOR⁵, CONR⁶R⁷, SO₂R⁵ and SO₂NR⁶R⁷, or is naphthyl or naphthyl substituted by one or more substituents selected from
   (C₁-C₄)-alkyl, (C₁-C₄)-alkoxy, CF₃, NO₂, CN, halogen, COR⁵, COOR⁵, CONR⁶R⁷, SO₂R⁵ and SO₂NR⁶R⁷,
      for dyeing and printing hydrophobic synthetic materials.

Useful hydrophobic synthetic materials include for example secondary cellulose acetate, cellulose triacetate, polyamides and, in particular, high molecular weight polyesters. Materials composed of high molecular weight polyesters are in particular those based on polyethylene glycol terephthalates.

The hydrophobic synthetic materials can be present in the form of sheet- or threadlike constructions and can have been processed, for example, into yarns or into woven or knitted textile materials Fibrous textile materials are preferred. Polyester fibers and polyester textile materials for automotive fabrics are very particularly preferred.

A preferred embodiment of the use according to the present invention comprises utilizing dye mixtures comprising at least one dye of the general formula I and at least one dye useful for dyeing polyester fibers and polyester textile materials for automotive fabrics.

The dyeing in accordance with the use provided by the present invention can be carried out in a conventional manner, preferably from an aqueous dispersion, if appropriate in the presence of carriers, at between 80 to about 110° C. by the exhaust process or by the HT process in a dyeing autoclave at 110 to 140° C., and also by the so-called thermofix process, in which the fabric is padded with the dyeing liquor and subsequently fixed/set at about 180 to 230° C.

Printing of the materials mentioned can be carried out in a manner known per se by incorporating the dye or dye mixtures of the present invention in a print paste and treating the fabric printed therewith at temperatures between 180 to 230° C. with HT steam, high-pressure steam or dry heat, if appropriate in the presence of a carrier, to fix the dye.

The dyes and dye mixtures of the present invention are very particularly useful for dyeing and printing polyester fibers and polyester textile materials for automotive fabrics. It is preferable for the dyeing and printing to be carried out in the presence of UV absorbers, for example UV absorbers based on benzophenone or benzotriazole. Details concerning the dyeing and printing of automotive fabrics are known to those skilled in the art and are described in the pertinent literature.

In addition, however, the dyes and dye mixtures of the present invention can also be used for dyeing and printing hydrophobic synthetic materials envisaged for other purposes, examples being alkalized polyester fibers, polyester microfibers or materials that are not in fiber form.

The dyes and dye mixtures of the present invention shall be in a very fine state of subdivision when they are used in dyeing liquors, padding liquors or print pastes.

The dyes are converted into the fine state of subdivision in a conventional manner by slurrying the as-fabricated dye together with dispersants in a liquid medium, preferably in water, and subjecting the mixture to the action of shearing forces to mechanically comminute the original dye particles to such an extent that an optimal specific surface area is achieved and sedimentation of the dye is minimized. This is accomplished in suitable mills, such as ball or sand mills. The particle size of the dyes is generally between 0.5 and 5 μm and preferably equal to about 1 μm.

The dispersants used in the milling operation can be nonionic or anionic. Nonionic dispersants include for example reaction products of alkylene oxides, for example ethylene oxide or propylene oxide, with alkylatable compounds, for example fatty alcohols, fatty amines, fatty acids, phenols, alkylphenols and carboxamides. Anionic dispersants are for example lignosulfonates, alkyl- or alkylarylsulfonates or alkylaryl polyglycol ether sulfates.

The dye preparations thus obtained should be pourable for most applications. Accordingly, the dye and dispersant content is limited in these cases. In general, the dispersions are adjusted to a dye content up to 50 percent by weight and a dispersant content up to about 25 percent by weight. For economic reasons, dye contents are in most cases not allowed to be below 15 percent by weight.

The dispersions may also contain still further auxiliaries, for example those which act as an oxidizing agent, for example sodium m-nitrobenzenesulfonate, or fungicidal agents, for example sodium o-phenylphenoxide and sodium pentachlorophenoxide, and particularly so-called ‘acid donors’, examples being butyrolactone, monochloroacetamide, sodium chloroacetate, sodium dichloroacetate, the sodium salt of 3-chloropropionic acid, monosulfate esters such as lauryl sulfate for example, and also sulfuric esters of ethoxylated and propoxylated alcohols, for example butylglycol sulfate.

The dye dispersions thus obtained are very advantageous for making up dyeing liquors and print pastes.

There are certain fields of use where powder formulations are preferred. These powders comprise the dye or dye mixture, dispersants and other auxiliaries, for example wetting, oxidizing, preserving and dustproofing agents and the abovementioned “acid donors”.

A preferred method of making pulverulent preparations of dye consists in stripping the above-described liquid dye dispersions of their liquid, for example by vacuum drying, freeze drying, by drying on drum dryers, but preferably by spray drying.

The dyeing liquors are made by diluting the requisite amounts of the above-described dye formulations with the dyeing medium, preferably water, such that a liquor ratio of 5:1 to 50:1 is obtained for dyeing. In addition, it is generally customary to include further dyeing auxiliaries, such as dispersing, wetting and fixing auxiliaries, in the liquors. Organic and inorganic acids such as acetic acid, succinic acid, boric acid or phosphoric acid are included to set a pH in the range from 4 to 5, preferably 4.5. It is advantageous to buffer the pH setting and to add a sufficient amount of a buffering system. The acetic acid/sodium acetate system is an example of an advantageous buffering system.

To use the dye or dye mixture in textile printing, the requisite amounts of the abovementioned dye formulations are kneaded in a conventional manner together with thickeners, for example alkali metal alginates or the like, and if appropriate further additives, for example fixation accelerants, wetting agents and oxidizing agents, to give print pastes.

### EXAMPLE 1

8 parts of 6-aminoanthraquinone-2,1-acridone (general formula VIII where R¹-R⁴=hydrogen) are suspended in 100 parts of chlorobenzene. 4.5 parts of 4-chlorobutyryl chloride are added dropwise at 80-100° C. before stirring for 2 h under reflux, cooling down to room temperature, filtering off with suction and washing with chlorobenzene and then with methanol to obtain 10 parts of the dye of the formula Ia

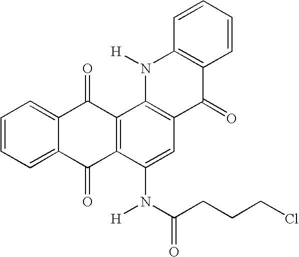

### EXAMPLE 2

50 parts of 6-aminoanthraquinone-2,1-acridone (general formula VIII where R¹-R⁴=hydrogen) are introduced as initial charge in 664 parts of chlorobenzene. 16 parts of pyridine and 23 parts of methanesulfonyl chloride are added at 100° C. The mixture is stirred at 125° C. for 15 h and is then admixed with a further 3.2 parts of pyridine and 4.6 parts of methanesulfonyl chloride. It is stirred at 125° C. for a further 12 h and then cooled down to room temperature. The product is filtered off with suction and washed with chlorobenzene and then with methanol to obtain 49 parts of the dye of the formula Ib

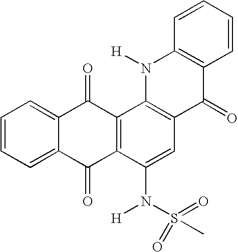

Example 2 is repeated to obtain the dyes in the table hereinbelow.

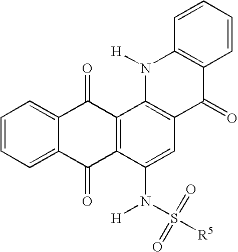

| Example | R⁵ |
|---|---|
| 3 | Et |
| 4 | n-Pr |
| 5 | i-Pr |
| 6 | n-Bu |
| 7 | 1-naphthyl |
| 8 | 2-naphthyl |
| 9 | phenyl |
| 10 | 4-methylphenyl |
| 11 | 4-chlorophenyl |
| 12 | 2-bromophenyl |
| 13 | 4-bromophenyl |
| 14 | 2-nitrophenyl |
| 15 | 3-nitrophenyl |
| 16 | 4-nitrophenyl |
| 17 | phenylmethyl |
| 18 | 4-chloro-3-nitrophenyl |
| 19 | 3-trifluoromethylphenyl |
| 20 | 3,4-dimethoxyphenyl |
| 21 | 4-methoxyphenyl |

### EXAMPLE 22

30 g of the water-moist presscake of the dye obtained according to Example 2 in 200 ml of water are admixed with 63 g of sodium lignosulfonate and 3 g of a nonionic dispersant (addition product of abietic acid and 50 mol equivalents of ethylene oxide) and adjusted to pH 7 with 25% sulfuric acid. This is followed by bead milling at room temperature for 1 h to 90%<1 μm, sieving and drying in a spray dryer. 2 g of the powder thus obtained are dispersed in 1000 g of water. The dispersion is admixed with 0.5 to 2 g per l of liquor of a commercially available dispersant based on a condensation product of naphthalenesulfonic acid sodium salt and formaldehyde, 0.5 to 2 g per l of liquor of monosodium phosphate and 2 g per l of liquor of a commercially available leveling assistant and adjusted to pH 4.5-5.5 with acetic acid. The dyeing liquor thus obtained is entered with 100 g of a woven fabric of textured polyester based on polyethylene glycol terephthalate before dyeing at 130° C. for 60 min. The blue dyeing obtained after reduction clearing possesses excellent lightfastness and colorfastness to light at high temperatures and very good fastness to sublimation.

Repeating this example with the dyes of Examples 1 and 3-21 likewise gives blue dyeings of excellent colorfastness to light at high temperatures.

### EXAMPLE 23

0.176 g of the dye of the formula 1a of Example 1 are dissolved in 10 ml of DMF with heating, and the solution is admixed with 1 ml of conc. Levegal® DLP (commercial product of Lanxess Deutschland GmbH) and also 290 ml of water.

While stirring, 0.318 g of the dye C.I. Disperse Yellow 71 (as 33.7% strength finished material) and 0.300 g of the dye C.I. Disperse Red 86 (as 34.9% strength finished material) are added. The pH is set to 4.5 with acetic acid/sodium acetate and 1 g of Levegal DLP are added per 1 l of this liquor, A volume containing 0.005 g of dye of the formula Ia of Example 1 is taken from this stock solution, made up to 100 ml with water and entered with 5 g of pile polyester fabric. Dyeing is carried out at 135° C. for 45 min using a heating-up rate of 1 degree/min. Cooling is followed by hot and cold rinsing. The gray dyeing obtained after reduction clearing possesses excellent colorfastness to light at high temperatures.

Repeating this dyeing in the presence of 0.100 g of a UV absorber based on phenyltriazines or benzotriazoles likewise gives gray dyeings of excellent colorfastness to light at high temperatures, with the colorfastness to light at high temperatures being somewhat higher in the case of the benzotriazole than without use of a UV absorber.

### EXAMPLE 24

0.150 g of the dye of Example 9 and 0.150 g of the dye of Example 10 are dissolved in 10 ml of DMF with heating, and the solution is admixed with 1 ml of conc. Levegal DLP and also 290 ml of water. While stirring, 0.388 g of the dye C.I. Disperse Yellow 71 (as 5.3% strength finished material), 0.388 g of the dye C.I. Solvent Yellow 163 (as 24.6% strength finished material) and 0.185 g of a dye of the formula IV where R¹⁴=phenyl and R¹⁵=hydroxyethoxyethyl in the form of the isomeric mixture with R¹⁴ and R¹⁵ (as 23.4% strength finished material) are added. The pH is set to 4.5 with acetic acid/sodium acetate and 1 g of Levegal DLP are added per 1 l of this liquor.

A volume containing 0.00425 g of dye of Example 9 is taken from this stock solution, made up to 100 ml with water and entered with 5 g of pile polyester fabric. Dyeing is carried out at 135° C. for 45 min using a heating-up rate of 1 degree/min. Cooling is followed by hot and cold rinsing. The gray dyeing obtained after reduction clearing possesses excellent colorfastness to light at high temperatures.

Repeating this dyeing in the presence of 0.150 g of a UV absorber based on phenyltriazines or of 0.100 g of a UV absorber based on benzotriazoles likewise gives gray dyeings of excellent colorfastness to light at high temperatures. In both cases, the colorfastness to light at high temperatures is somewhat higher than without use of a UV absorber, and with the phenyltriazine fading on tone is achieved in that the hue of the shade does not change in the course of fading.

We claim:

1. A dye of the general formula I 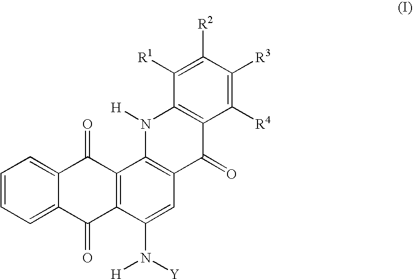
where
R¹ to R⁴ are independently hydrogen, (C₁-C₄)-alkyl, (C₁-C₄)-alkoxy, CF₃, NO₂, CN, halogen, COR⁵, COOR⁵CONR⁶R⁷, SO₂R⁵ or SO₂NR⁶R⁷
where R⁵, R⁶ and R⁷ are each hydrogen or (C₁-C₄)-alkyl, but R⁶ and R⁷ cannot both be hydrogen; and
Y is —CO(CH₂)₃Cl or —SO₂R⁸,
where R⁸ is (C₁-C₈)-alkyl, (C₁-C₈)-alkyl, substituted by NO₂, CN, halogen or phenyl, phenyl, phenyl substituted by one or more substituents selected from (C₁-C₄)-alkyl, (C₁-C₄)-alkoxy, CF₃, NO₂, CN, halogen, COR⁵, COOR⁵, CONR⁶R⁷, SO₂R⁵ and SO₂NR⁶R⁷, or is naphthyl or naphthyl substituted by one or more substituents selected from
(C₁-C₄)-alkyl, (C₁-C₄)-alkoxy, CF₃, NO₂, CN, halogen, COR⁵, COOR⁵, CONR⁶R⁷, SO₂R⁵ and SO₂NR⁶R⁷,
although R⁸ cannot be 4-methylphenyl when R¹ to R⁴ are all hydrogen and cannot be phenyl or 4-methylphenyl when R¹ and R³ are both chlorine and R² and R⁴ are both hydrogen.

2. A dye as claimed in claim 1, wherein R¹ to R⁴ are all hydrogen.

3. A dye as claimed in claim 1 and/or 2, wherein R⁸ is ethyl, n-propyl, i-propyl, n-butyl, 1-naphthyl, 2-naphthyl, phenyl, 4-methylphenyl, 4-chlorophenyl, 2-bromophenyl, 4-bromophenyl, 2-nitrophenyl, 3-nitrophenyl, 4-nitrophenyl, phenylmethyl, 4-chloro-3-nitrophenyl, 3-trifluoromethylphenyl, 3,4-dimethoxyphenyl or 4-methoxyphenyl.

4. A dye mixture comprising at least one dye of the general formula I according to claim 1 and at least one dye useful for dyeing polyester textile materials for automotive fabrics

5. A dye mixture as claimed in claim 4, wherein a dye useful for dyeing polyester textile materials for automotive fabrics is C.I. Disperse Yellow 23, 42, 51, 59, 65, 71, 86, 108, 122, 163, 182 and 211, C.I. Solvent Yellow 163, C.I. Disperse Orange 29, 30, 32, 41, 44, 45, 61 and 73, C.I. Pigment Orange 70, C.I. Solvent Brown 53 or a dye of the formulae II or III 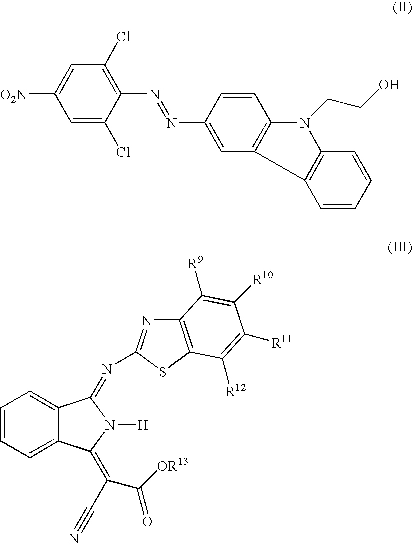
where
R⁹ to R¹² are independently hydrogen, chlorine, methyl, ethyl, isopropyl, tert-butyl, cyclohexyl, methoxy, ethoxy, n-propoxy, n-butoxy, methoxyethyl, ethoxyethyl, butoxyethyl or phenoxy, and
R¹³ is methyl, ethyl, propyl, isopropyl, allyl, n-butyl, isobutyl, n- and isopentyl, hexyl, octyl, 2-ethylhexyl, methoxyethyl, ethoxyethyl, butoxyethyl, butoxyethoxyethyl.

6. A dye mixture as claimed in claim 4, wherein a dye useful for dyeing polyester textile materials for automotive fabrics is C.I. Disperse Red 60, 82, 86, 91, 92, 127, 134, 138, 159, 167, 191, 202, 258, 279, 284, 302 and 323, C.I. Solvent Red 176 or a dye of the formulae IV, V or VI 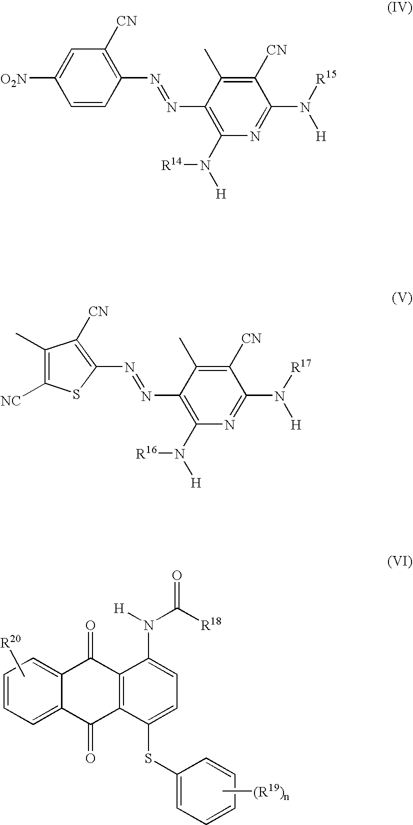
where
R¹⁴ and R¹⁵ are independently hydroxyethoxyethyl or phenyl,
R¹⁶ and R¹⁷ are independently hydrogen, hydroxyethoxyethyl, hydroxybutoxypropyl, acetoxyethoxyethyl or acetoxybutoxypropyl,
R¹⁸ is (C₁-C₈)-alkyl, phenyl or phenyl substituted by (C₁-C₄)-alkyl, hydroxyl or halogen, and
R¹⁹ and R²⁰ are independently hydrogen or halogen, and also
n is 0, 1 or 2.

7. A dye mixture as claimed in claim 4, wherein a dye useful for dyeing polyester textile materials for automotive fabrics is C.I. Blue 27, 54, 56, 60, 73, 77, 79, 79:1, 87, 266, 333 and 361, C.I. Disperse Violet 27, 28, 57 and 95 or a dye of the formula VII 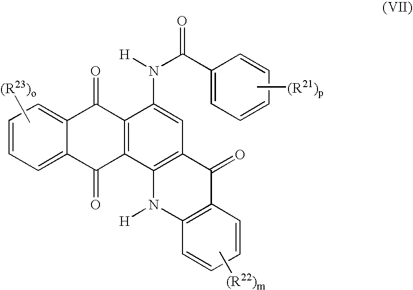
where
R²¹, R²² and R²³ are independently (C₁-C₈)-alkyl, halogen or hydroxyl, and m, o and p are independently 0, 1 or 2.

8. A process for preparing a dye of the general formula I according to claim 1, which comprises reacting a compound of the general formula VIII 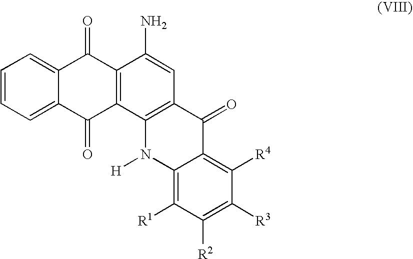
where R¹ to R⁴ are each as defined in claim 1, with a compound of the general formula IX
Hal-Y (IX)
where Hal is halogen, particularly chlorine, and Y is as defined in claim 1.

9. The use of a dye of the general formula I 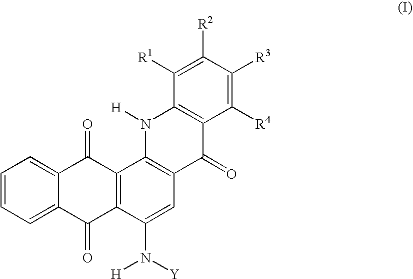
where
R¹ to R⁴ are independently hydrogen, (C₁-C₄)-alkyl, (C₁-C₄)-alkoxy, CF₃, NO₂, CN, halogen, COR⁵, COOR⁵, CONR⁶R⁷, SO₂R⁵ or SO₂NR⁶R⁷,
where R⁵, R⁶ and R⁷ are each hydrogen or (C₁-C₄)-alkyl, but R⁶ and R⁷ cannot both be hydrogen; and
Y is —CO(CH₂)₃Cl or —SO₂R⁸,
where R⁸ is (C₁-C₈)-alkyl, (C₁-C₈)-alkyl, substituted by NO₂, CN, halogen or phenyl, phenyl, phenyl substituted by one or more substituents selected from (C₁-C₄)-alkyl, (C₁-C₄)-alkoxy, CF₃, NO₂, CN, halogen, COR⁵, COOR⁵, CONR⁶R⁷, SO₂R⁵ and SO₂NR⁶R⁷, or is naphthyl or naphthyl substituted by one or more substituents selected from
(C₁-C₄)-alkyl, (C₁-C₄)-alkoxy, CF₃, NO₂, CN, halogen, COR⁵, COOR⁶, CONR⁶R⁷, SO₂R⁵ and SO₂NR⁶R⁷,
for dyeing and printing hydrophobic synthetic materials.

10. The use as claimed in claim 9, wherein polyester fibers and polyester textile materials for automotive fabrics are utilized as hydrophobic synthetic materials.
